(19) United States
(12) Patent Application Publication
(10) Pub. No.: US 2024/0207240 A1
(43) Pub. Date: Jun. 27, 2024

(54) Method for Improving or Treating Cognitive Dysfunction Complicating HIV-associated Cryptococcal Meningitis with Lenalidomide

(71) Applicant: The First Affiliated Hospital Zhejiang University School of Medicine, Hangzhou (CN)

(72) Inventors: Biao Zhu, Hangzhou (CN); Ran Tao, Hangzhou (CN); Ye Xiong, Hangzhou (CN); Xiaorong Peng, Hangzhou (CN); Ying Huang, Hangzhou (CN)

(21) Appl. No.: 18/391,645

(22) Filed: Dec. 20, 2023

Related U.S. Application Data
(60) Provisional application No. 63/434,101, filed on Dec. 21, 2022.

(51) Int. Cl. A61K 31/454; A61K 45/06; A61P 25/28

(52) U.S. Cl. CPC A61K 31/454; A61K 45/06; A61P 25/28

(57) ABSTRACT

The present invention provides a method for treating patients with cognitive dysfunction complicating HIV-associated CM with lenalidomide. Patients with HIV-associated CM develop IRIS, some of which develop cognitive dysfunction. The patients with HIV-associated CM-IRIS are diagnosed as having cognitive dysfunction by Chinese version of the Montreal Cognitive Assessment (MoCA) and International HIV Dementia Scale (IHDS). After the treatment with lenalidomide, the MoCA score and IHDS score of the patients are improved significantly, and the leukocyte, proteins, albumin, IgG and inflammatory cytokines (growth-related oncogene, interleukin [IL]-10, granulocyte-colony stimulating factor, IL-6, IL-8, complement factor H, tumor necrosis factor-α, and α2 macroglobulin) in cerebrospinal fluid are greatly reduced. The present invention widens the scope of application of lenalidomide, proposes a new treatment method for cognitive dysfunction caused by HIV-associated CM-IRIS, and provides a new idea for the research and development of new drugs for cognitive dysfunction caused by HIV-associated CM-IRIS.

### CROSS-REFERENCE TO RELATED APPLICATION

The present application claims priority from U.S. Provisional Patent Application No. 63/434,101 filed on Dec. 21, 2022, the contents of which are incorporated herein by reference in their entirety.

### TECHNICAL FIELD

The present invention relates to the field of medicines, and particularly to a method for preventing or treating cognitive dysfunction caused by human immunodeficiency virus (HIV)-associated cryptococcal meningitis (CM)-immune reconstitution inflammatory syndrome (IRIS).

### BACKGROUND

CM is a common opportunistic infection in patients with acquired immunodeficiency syndrome (AIDS) and is responsible for 15% of all HIV-related mortality. Some patients might experience deterioration in neurological status with an increasing CD4+ T-cell count and a decreasing HIV-RNA load in the peripheral blood after antiretroviral therapy (ART) and antifungal regimens. However, few studies on the cause and treatment of this cognitive dysfunction have been performed

Currently, it is postulated that cognitive dysfunction might be associated with paradoxical immune reconstitution inflammatory syndrome (IRIS). IRIS is a common complication of cryptococcal infection, including unmasking CM-IRIS and paradoxical IRIS. Unmasking IRIS develops in previously undiagnosed CM in ART-naïve individuals, while paradoxical IRIS occurs in patients previously treated for CM with cryptococcal sterilization in cerebrospinal fluid (CSF) following the initiation of highly active ART (HAART). Worsening cognitive dysfunction and clinical manifestation frequently present in HIV-associated CM (HCM) patients with paradoxical IRIS. It has been reported that approximately 25% of HCM patients develop CM-IRIS in the first 4 months of ART, with an average mortality of 20±10%. Thus, an effective treatment for IRIS would be helpful to decrease the likelihood of cognitive dysfunction.

Recent studies have found an imbalance between the proinflammatory Th1/Th17 and anti-inflammatory Th2/Treg axes in paradoxical IRIS followed by a hyperactive compensatory Th1 response, which leads to an inflammatory burst and aggravation of inflammation in the CSF compartment, manifesting as a deterioration in neurological status, such as cognitive dysfunction.

Short courses of corticosteroids are the mainstay of treatment of IRIS; however, the limitations of corticosteroid treatment should not be ignored. The long-term use of corticosteroid will lead to osteoporosis, neuropsychiatric manifestations, aseptic joint necrosis, adrenal insufficiency, gastrointestinal diseases and increased risk of other infections. In the previous study, although corticosteroid therapy was used to treat IRIS, it was not successful, indicating that corticosteroid therapy has the risk of injuring patients. Consequently, optimal therapeutics for cognitive dysfunction are urgently needed in clinical practice. In recent years, thalidomide and its derivatives (lenalidomide and pomalidomide), termed immunomodulatory imide drugs (IMiDs), have been used in neurodegenerative disease due to their high blood-brain barrier permeability and bioavailability.

The use of thalidomide in the treatment of HIV-IRIS has also been reported. Its side effects, such as bone marrow suppression, cardiotoxicity, vascular thrombosis, and neurotoxicity, limit its usage in clinical application. Lenalidomide, as a new-generation IMiD drug, has an inhibitory effect on TNF-α that is 2000 times that of thalidomide. Moreover, the side effects of lenalidomide, such as constipation, peripheral neuropathy, sleepiness and neurotoxicity, are milder than those of thalidomide.

Lenalidomide has been studied in the treatment of central system diseases and HIV-related diseases, such as central nervous system tumors, POEMS syndrome, neuropathy, relapsed AL amyloidosis, Kaposi sarcoma and pseudotumoral herpes simplex virus type 2 infection in HIV infection. However, its use in cognitive dysfunction has never been reported in HCM patients with IRIS.

### SUMMARY

The present invention provides a method for treating cognitive dysfunction caused by HIV-associated CM-IRIS with lenalidomide, to effectively improve cognitive dysfunction in such patients. The method includes cognitive assessment during the treatment of cognitive dysfunction caused by HIV-associated CM-IRIS with lenalidomide, and the changes of biochemistry and cytokines in cerebrospinal fluid of patients after treatment with lenalidomide.

The method for improving or treating cognitive dysfunction complicating HIV-associated CM in a patient with lenalidomide includes administering a therapeutically effective amount of lenalidomide to the patient.

Lenalidomide is administered at 25 mg/day for immuno-modulation for a total of 6 treatment cycles, with each cycle including 21 days of lenalidomide administration followed by 7 days of no lenalidomide.

Preferably, the median time between ART initiation and lenalidomide initiation is 310.0-710.5 days.

Preferably, the treatment with lenalidomide is carried out on the basis of continuing ART and anti-cryptococcal treatment.

The treatment is directed to patients who have cryptococcosis diagnosed before ART; have completed 2 weeks of induction and 8 weeks of consolidation treatment or are negative for two times of CSF cryptococcus culture; or have plasma HIV-1 RNA of less than 500 copies per milliliter; and patients with chronic inflammation of the central system.

The patients with chronic inflammation of the central system have one or more of:
1. CSF protein of higher than 0.45 g/L
2. CSF nucleated cell count of greater than 8/uL
3. lesions in the head indicated by abnormal signals in radiological examination, such as inflammation and edema etc.

The treatment is not intended for patients treated with immunosuppressants or other immunomodulators or cytotoxic drugs within 6 months.

The treatment is not intended for patients with severe underlying diseases of the heart, brain, liver, and kidney,

The treatment is not intended for patients with absolute neutrophil count of 1000 cells/uL or less and a platelet count of less than 75,000/uL.

Preferably, before the treatment with lenalidomide, the patients receive antifungal treatments, complete the initial therapy of CM with amphotericin B (AmB) and 5-flucytosine (5-FC), and have negative CSF fungal cultures; and then they are continuously treated with fluconazole (FLU). The induction treatment comprises AmB (0.7-1.0 mg/kg)+5-FC (100 mg/kg bid) for 2 weeks. The consolidation treatment comprises 800 mg FLU for 8 weeks, and the maintenance treatment comprises 200 mg FLU daily.

The treatment reduces leukocytes, proteins, albumin, IgG and inflammatory cytokines in CSF. The inflammatory cytokines include growth-related oncogene, interleukin [IL]-10, granulocyte-colony stimulating factor, IL-6, IL-8, complement factor H, tumor necrosis factor-α, and a2 macroglobulin.

The present invention further provides a drug for treating cognitive dysfunction caused by HIV-associated CM-IRIS, in which the active component includes lenalidomide.

Patients with HIV-associated CM develop IRIS, some of which develop cognitive dysfunction. The patients are diagnosed as having cognitive dysfunction by Chinese version of the Montreal Cognitive Assessment (MoCA) and International HIV Dementia Scale (IHDS). After the treatment with lenalidomide, the MoCA score and IHDS score of the patients are improved significantly, that is, the cognitive dysfunction in the patients is effectively improved, and the leukocyte, proteins, albumin, IgG and inflammatory cytokines (growth-related oncogene, interleukin [IL]-10, granulocyte-colony stimulating factor, IL-6, IL-8, complement factor H, tumor necrosis factor-α, and α2 macroglobulin) in CSF are greatly reduced. The present invention widens the scope of application of lenalidomide, proposes a new treatment method for cognitive dysfunction caused by HIV-associated CM-IRIS, and provides a new idea for the research and development of new drugs for cognitive dysfunction caused by HIV-associated CM-IRIS.

### BRIEF DESCRIPTION OF THE DRAWINGS

FIGS. 1A and 1B compare the cognitive assessment scores of patients with cognitive dysfunction caused by HIV-associated CM-IRIS before and after treatment with lenalidomide. The patients with HIV-associated CM-IRIS are diagnosed as having cognitive dysfunction by Chinese version of the Montreal Cognitive Assessment (MoCA) and International HIV Dementia Scale (IHDS). On the basis of continuing ART and anti-cryptococcal treatment, lenalidomide is administered at 25 mg/day for immuno-modulation for a total of 6 treatment cycles, with each cycle including 21 days of lenalidomide administration followed by 7 days of no lenalidomide. FIG. 1A compares the MoCA and IHDS scores of the patients before and after treatment. FIG. 1B compares the scores of seven domains in the MoCA scale of the patients before and after treatment.

FIG. 1C compares the scores of three items in the IHDS scale of the patients before and after treatment.

The median MoCA score improved by three points after lenalidomide treatment [WO: 23.0 (13.0-24.0) vs W24: 26.0 (24.0-28.0), P=0.018]. Five patients had IHDS scores <10 at baseline. There was also a significant improvement in IHDS scores after lenalidomide treatment [WO: 9.0 (2.5-10.5) vs W24: 11.0 (10.0-12.0), P=0.028] (FIG. 1A). Improvements were mainly observed memory in the MoCA [WO: 1.0 (0-3.0) vs W24: 4.0 (3.0-5.0), P=0.026] and in recall of the four items in the IHDS [WO: 1.5 (1.0-3.5) vs W24: 4.0 (2.0-4.0), P=0.042] (FIG. 1B) (FIG. 1C).

FIGS. 2A and 2B show results of treating patients with HIV-associated CM who still develop cognitive dysfunction after completing the anti-cryptococcal induction treatment. The patients are treated with lenalidomide. In the treatment, lenalidomide is administered at 25 mg/day for immuno-modulation for a total of 6 treatment cycles, with each cycle including 21 days of lenalidomide administration followed by 7 days of no lenalidomide. The patients are further assigned to a cognitive improvement group and a cognitive non-improvement group. After assessment by the MoCA and IHDS scales, patients having a cognitive status that returns to the normal level after 6 weeks of treatment with lenalidomide are included in the cognitive improvement group, and patients having a cognitive status that does not return to the normal level are included in the cognitive non-improvement group. FIG. 2A compares CSF biochemistry in the cognitive improvement group and cognitive non-improvement group before and after treatment. FIG. 2B shows the CSF cytokine signaling pathway in the cognitive improvement group before and after treatment.

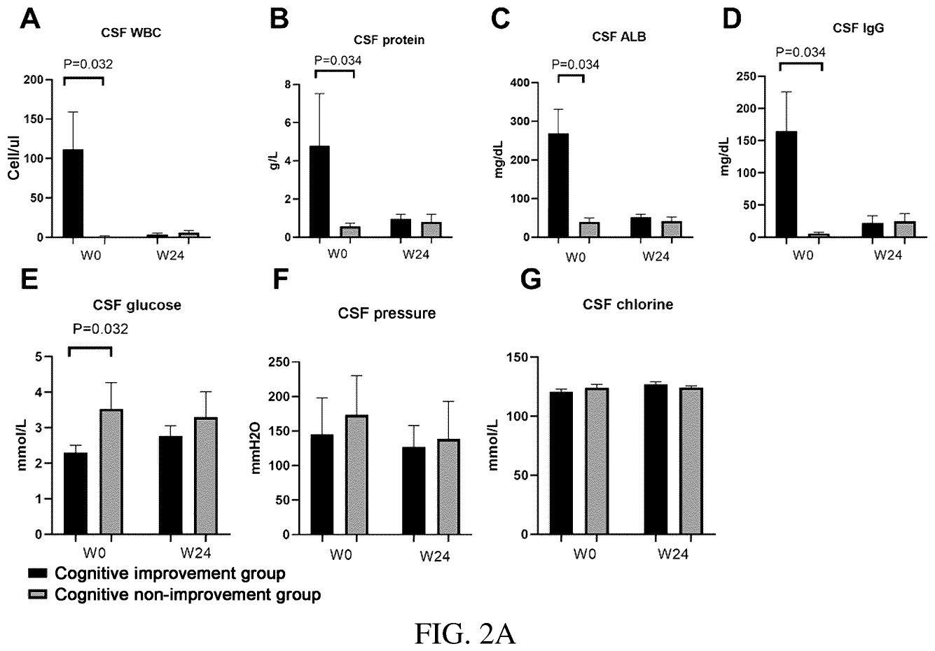

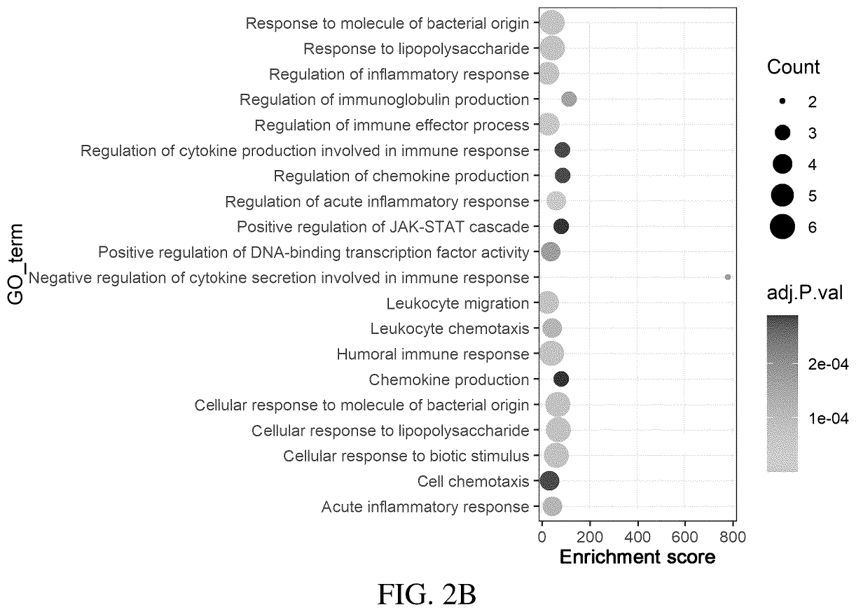

The CI group had a more inflammatory milieu before lenalidomide treatment, with a higher level of CSF white blood cells (WBCs) [x106/L; CI group: 94.0 (44.0-180.0) vs CNI group: 0 (0-1.5), P=0.032], CSF protein [g/L; CI group: 4.9 (3.0-6.6) vs CNI group: 0.6 (0.5-0.7), P=0.034], CSF albumin [g/L; CI group: 318.5 (190.9-346.5) vs CNI group: 33.5 (30.4-46.2), P=0.034], and CSF IgG [g/L; CI group: 160.5 (73.8-256. 0) vs CNI group: 4.7 (4.3-7.4), P=0.034]. CSF glucose levels were much lower in the CI group than in the CNI group [mmol/L; CI group: 2.4 (2.0-2.7) vs CNI group: 2.8 (2.8-3.9), P=0.032] (A-E in FIG. 2A). Furthermore, there was no significant difference in CSF pressure or CSF chlorine levels between the two groups (F-G in FIG. 2A).

The cytokine signaling pathways that changed significantly after treatment in the CI group were mainly in the cellular response to lipopolysaccharide, regulation of acute inflammatory response, etc (FIG. 2B).

### DETAILED DESCRIPTION

Unless otherwise specified, the reagents used in this application are all commercially available, and lenalidomide is purchased from Qilu Pharmaceutical Co., Ltd. (Qipuyi® lenalidomide).

Study design and patient inclusion criteria: This is an open-label, single-center, prospective observational study conducted in the First Affiliated Hospital of Zhejiang University.

Inclusion criteria
1. HIV-1-infected individuals
2. those having cryptococcosis diagnosed before ART (positive culture, or typical clinical features that are positive for India ink staining, or antigen detection positive)
3. those experiencing successful induction treatment for AIDS-related CM (including the following two situations):
   A. those completing 2 weeks of induction and 8 weeks of consolidation treatment
   B. those having two times of negative cerebrospinal fluid (CSF) cryptococcus culture;
4. those receiving ART
5. those having plasma HIV-1 RNA of less than 500 copies per milliliter
6. patients with chronic inflammation of the central system and having one or more of:
   1. CSF protein of higher than 0.45 g/L
   2. CSF nucleated cell count of greater than 8/uL
   3. lesions in the head indicated by abnormal signals in radiological examination, such as inflammation and edema etc
7. patients with cognitive dysfunction
8. not drinking or smoking
9. willing to give informed consent and sign the informed consent form
   Exclusion criteria:
1. those who are pregnant or breastfeeding
2. patients having poor treatment compliance
3. patients treated with immunosuppressants or other immunomodulators or cytotoxic drugs within 6 months before screening,
4. patients with severe underlying diseases of the heart, brain, liver, and kidney,
5. those with an absolute neutrophil count of 1000 cells per uL or less and a platelet count of less than 75,000/uL
6. those with known hypersensitivity or contraindication to lenalidomide,
7. those receiving other clinical trials in 3 months

Definition of cognitive dysfunction: The Chinese version of the Montreal Cognitive Assessment (MoCA) shown in Table 1, International HIV Dementia Scale (IHDS) shown in Table 2, and Center for Epidemiological Research Depression Scale (CES-D) are used to assess patients' cognitive status. They all use Chinese questionnaires that have been validated for the evaluation of Chinese patients. The MoCA tests eight major neurocognitive domains, including short-term memory recall (no score), visuospatial ability, naming, attention, language, conceptual thinking, working memory, and orientation of time and place. The MoCA, with a sensitivity of 90% and a specificity of 87%, is used to identify patients with mild cognitive dysfunction. The IHDS measures timed fingertapping, timed alternating hand sequence, and recall of four items. It has a sensitivity of 80% and a specificity of 55%. The CES-D is a 20-item questionnaire that is used to assess the frequency of depression symptoms within the first 2 weeks of the study. A MoCA score <26 and/or an IHDS score <10 indicate cognitive dysfunction. A total CES-D score ≥16 indicates a clinically significant depressive state.

IRIS definition criteria (ACTG, 2009): The IRIS definition criteria are as follows: an increase in CD4 count after ART (≥50 cells/uL or a ≥ 2-fold increase) and/or virologic suppression (>0.5 logo decrease in plasma HIV viremia) is observed, but clinical symptoms consistent with an infectious or inflammatory condition, which cannot be explained by a newly acquired infection, an expected course of a previously recognized infection, or side effects of medications, are observed.

Screening process: 14 patients were included in the lenalidomide treatment cohort based on the inclusion and exclusion criteria: 8 had cognitive dysfunction at the time of enrollment. Among them, one patient withdrew from the study due to skin allergy, so only 7 patients were followed up for 24 weeks.

Therapeutic approaches: Antifungal Treatment: All patients had already completed initial therapy of CM with amphotericin B (AmB) and 5-flucytosine (5-FC) and had negative CSF fungal cultures; and had negative CSF fungal cultures; and they continued treatment with fluconazole (FLU). The induction treatment comprised AmB (0.7-1.0 mg/kg)+5-FC (100 mg/kg bid) for 2 weeks. The consolidation treatment comprised 800 mg FLU for 8 weeks, while the maintenance treatment comprised 200 mg FLU daily.

Lenalidomide Treatment: Patients were treated with six cycles of lenalidomide (one cycle: 25 mg/dayay for 3 weeks, followed by 1 week ceased lenalidomide) when they were enrolled. The drug was stopped at the end of 6 cycles.

Follow-up time: The subjects were followed up at 0, 1, 2, 3 and 6 cycles of lenalidomide treatment. During each follow-up session, blood and CSF examinations were performed. Blood examination included a full blood count, liver and kidney function tests, assessment of coagulation function, C-reactive protein, CD4+T lymphocyte cell count, and HIV viral load, as well as other pathogenic tests, such as polymerase chain reaction testing for Epstein-Barr (EB) virus DNA, cytomegalovirus DNA, and T-SPOT test for Mycobacterium tuberculosis. CSF was sent for routine analysis, including biochemistry, bacterial and fungal culture, EB virus DNA, cytomegalovirus DNA, India ink staining, cryptococcus antigen detection, and cytokine analysis. Cognitive evaluations were performed at baseline and after 2 and 6 cycles of lenalidomide.

Detection of inflammatory cytokines in CSF: CSF samples were collected from the participants and centrifuged immediately; and the supernatant was stored at −80° C. until testing. 32 CSF cytokines were analyzed using the Human Cytokine/Chemokine/Growth Factor Panel A (Merck Millipore, St. Louis, USA): α-2-macroglobulin (α-2-M), apolipoprotein A-I (apoA-I), Apo-E, complement C3, complement factor H (CFH), fibroblast growth factor 2 (FGF-2), granulocyte-colony stimulating factor (G-CSF), granulocyte-macrophage colony-stimulating factor (GM-CSF), interferon-γ (IFN-γ), growth-regulated oncogene (GRO), soluble CD40 ligand (sCD40L), platelet-derived growth factor-AA (PDGF-AA), interleukin (IL)-12p40, IL-12p70, IL-13, IL-1α, IL-1β, IL-2, IL-3, IL-4, IL-6, IL-8, IL-10, IL-17α, monocyte chemoattractant protein-1 (MCP-1), macrophage inflammatory protein-la (MIP-la), MIP-1β, tumor necrosis factor-a (TNF-α), IL-17α, amyloid-β (1-42) (αβ42), total Tau (tTau) and phosphorylated Tau 181 (pTau181), The abbreviations of CSF cytokines are provided in Table 3. The cytokine concentrations are expressed in pg/mL pre and post-lenalidomide therapy (the specific values are shown in Table 4).

### Experimental Results

Baseline data: The patients' demographic data are shown in (Table 5). As shown, all 7 patients are Chinese men, with an average age of 37.0 (interquartile range [IQR] 33.0-39.0 years). Of the 7 patients, 85.7% have at least a junior high school education. Most of the patients (71.4%) are treated with an antiviral regimen containing integrase inhibitors. The average CD4 count is 151.5 (119.3-239.0) cells/u L. The median HIV RNA copy number is 0 (0-447.0) copies/mL. The median time for cognitive dysfunction onset after ART initiation is 309.0 (265.0-694.0) days. The median time between ART initiation and lenalidomide initiation is 440.5 (310.0-710.5) days. Headache (4 patients) and alteration of consciousness (4 patients) are the most common clinical symptoms (Table 5). All patients receive standard anticryptococcal treatment as mentioned in the “Therapeutic approaches”, and no pathogens are detected in the CSF. All patients are treated with corticosteroids, but their cognitive function is not significantly improved. They are not treated with corticosteroids in the last 6 months.

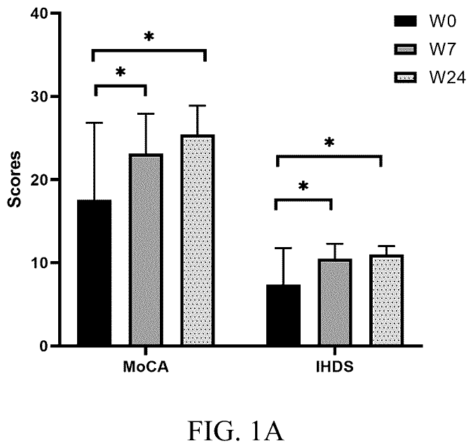

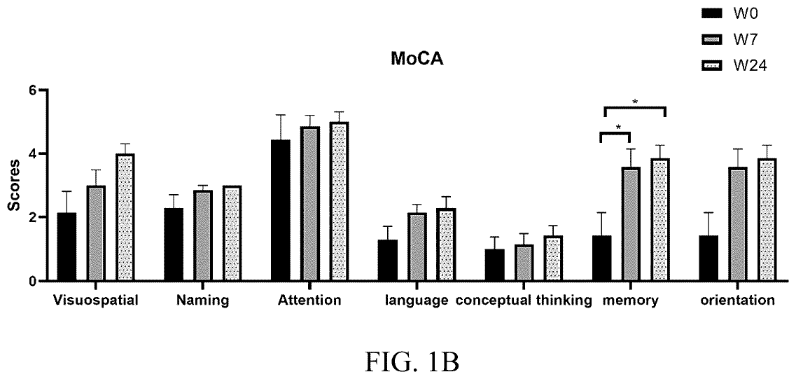

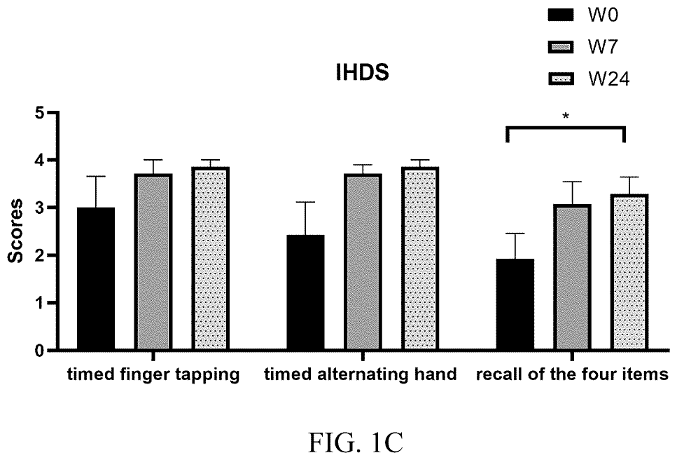

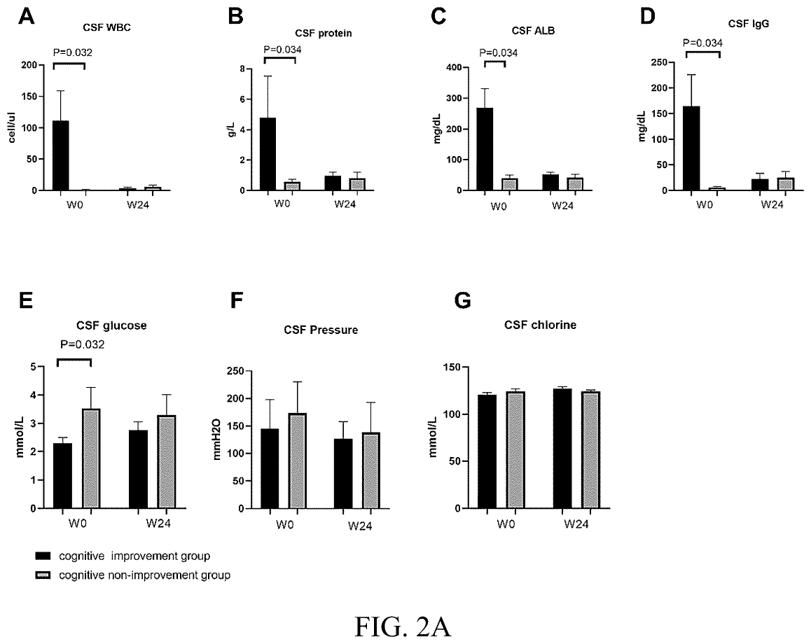

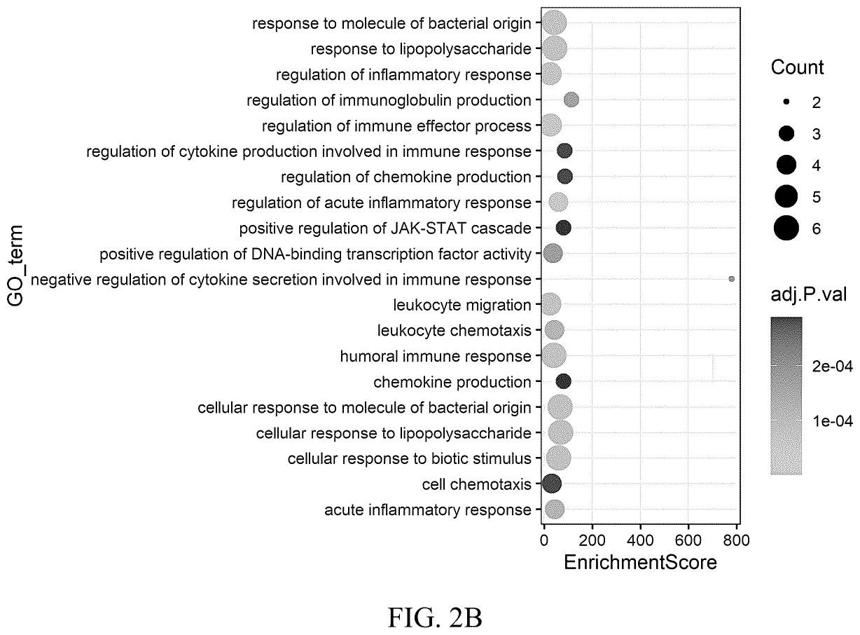

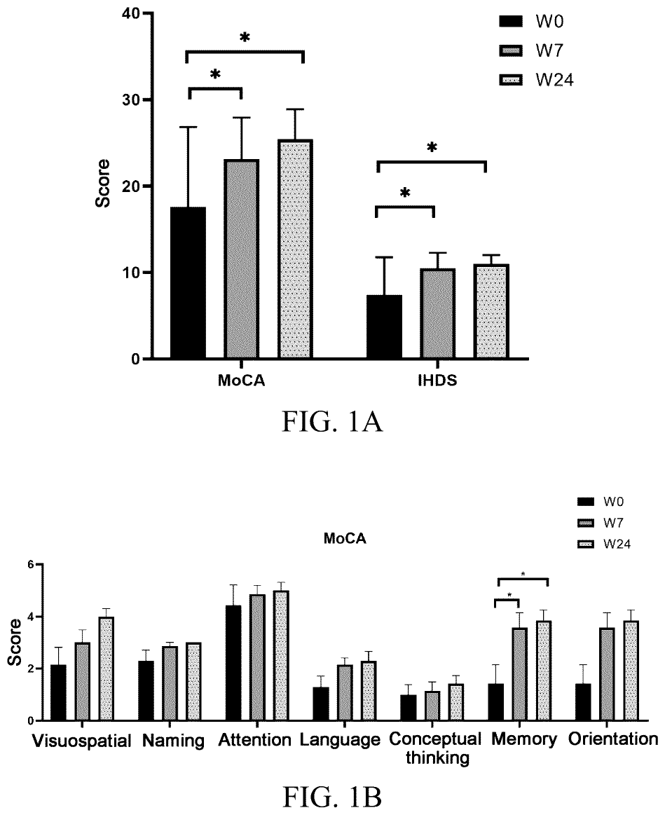

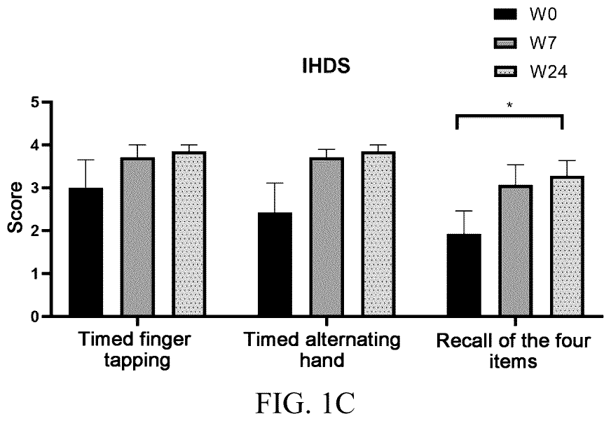

Cognitive improvement analysis: 6 patients have MoCA scores <26 at baseline. 5 patients have IHDS scores <10 at baseline. As shown in FIGS. 1A-1C, the median MoCA score is improved by 3 points after lenalidomide treatment [WO: 23.0 (13.0-24.0) vs W24: 26.0 (24.0-28.0), P=0.018], and there is a significant improvement in IHDS scores after lenalidomide treatment [WO: 9.0 (2.5-10.5) vs W24: 11.0 (10.0-12.0), P=0.028] (FIG. 1A). Improvements are mainly observed in memory in the MoCA [WO: 1.0 (0-3.0) vs W24: 4.0 (3.0-5.0), P=0.026] (FIG. 1B) and recall of the four items in the IHDS [WO: 1.5 (1.0-3.5) vs W24: 4.0 (2.0-4.0), P=0.042] (FIG. 1C). There is no significant difference in the CES-D score for anxiety or depression from before to after treatment. The two authoritative cognitive function assessment tests show that the lenalidomide treatment can significantly improve the cognitive function of patients. Spearman's correlation analysis shows a positive correlation between the MoCA score and IHDS score (R2=0.753, P<0.001). Furthermore, there is a negative correlation trend of CES-D scores with MoCA (R2=−0.349, P=0.029) and IHDS scores (R2=−0.498, P=0.001). This suggests that the results of the two cognitive function assessments are consistent.

Cognitive grouping analysis: Further analyses are performed for 7 patients. Among them, the cognitive status of 4 patients return to normal [the cognitive improvement group (CI group)], while that of 3 patients do not [the cognitive non-improvement group (CNI group)].

The pretreatment cognitive levels of the two groups are similar. The differences in the MoCA score abnormalities [CI group: 3/4 (75.0%) patients vs CNI group: 3/3 (100.0%) patients, P=0.265] and IHDS score abnormalities [CI group: 3/4 (75.0%) patients vs CNI group: 2/3 (66.7%) patients, P=0.462] between the two groups are not significant. However, after lenalidomide treatment, the MoCA scores of the patients in the CI group are improved significantly, and the proportion of patients with abnormal MoCA scores is significantly lower [CI group: 0/4 (0%) vs CNI group: 3/3(100%), P=0.002]. Meanwhile, the difference in the IHDS score between the two groups is not significantly different

CSF is further analyzed in both groups: the CI group has a more severe intracranial inflammation before lenalidomide treatment (FIGS. 2A-2B), with a higher level of CSF white blood cells (WBCs) [×106/L; CI group: 94.0 (44.0-180.0) vs CNI group: 0 (0-1.5), P=0.032], CSF protein [g/L; CI group: 4.9 (3.0-6.6) vs CNI group: 0.6 (0.5-0.7), P=0.034], CSF albumin [g/L; CI group: 318.5 (190.9-346.5) vs CNI group: 33.5 (30.4-46.2), P=0.034], and CSF IgG [g/L; CI group: 160.5 (73.8-256. 0) vs CNI group: 4.7 (4.3-7.4), P=0.034]. CSF glucose levels are much lower in the CI group than in the CNI group [mmol/L; CI group: 2.4 (2.0-2.7) vs CNI group: 2.8 (2.8-3.9), P=0.032] ((A-E) in FIG. 2A). Furthermore, there is no significant difference in CSF pressure or CSF chlorine levels between the two groups ((F-G) in FIG. 2A). After lenalidomide treatment, there is a significant decline in the CSF WBC (P=0.032), CSF protein (P=0.034), CSF albumin (P=0.034), and CSF IgG levels (P=0.050) in the CI group, showing that the lenalidomide treatment can significantly reduce intracranial inflammation in patients. Spearman's correlation analysis reveals that the decrease in CSF albumin level (R2: −0.757, P=0.049), and increase in CSF glucose level (R2: 0.883, P=0.008) have a strong positive correlation with MoCA scores (Table 6). The specific values are listed in Table 7. It is speculated that lenalidomide can improve the cognitive function in the patients by inhibiting intracranial inflammation.

CSF cytokine analysis: 32 cytokines are analyzed before and after lenalidomide treatment in the CI group and CNI group. In the CI group, there is a significant decline in cytokine levels after treatment (GRO, IL-10, G-CSF, IL-6, IL-8, P all <0.001; CFH, p=0.002; TNF-α, p=0.002; α-2-M, P=0.003). The actual values of the cytokines before and after lenalidomide treatment arc shown in Table 4. In the CNI group, only tTau levels are increased significantly (P=0.030). The cytokine signaling pathways that changed significantly after treatment in the CI group CI arc mainly in the cellular response to lipopolysaccharide, and regulation of acute inflammatory response, etc (FIG. 2B). It further shows that the effect of lenalidomide in the CI group is mainly in the regulation of inflammatory pathways. This may interpret the reason why lenalidomide is effective in the treatment of cognitive dysfunction.

In summary, the present invention provides a method for treating cognitive dysfunction caused by HIV-associated CM-IRIS with lenalidomide, to effectively improve cognitive dysfunction in such patients. The method includes cognitive assessment during the treatment of cognitive dysfunction caused by HIV-associated CM-IRIS with lenalidomide, and the changes of biochemistry and cytokines in cerebrospinal fluid of patients after treatment with lenalidomide. After the patients with cognitive dysfunction caused by HIV-associated CM-IRIS are administered and treated with lenalidomide, the cognitive dysfunction in the patients is effectively improved, and the leukocyte, proteins, albumin, IgG and inflammatory cytokines (growth-related oncogene, interleukin [IL]-10, granulocyte-colony stimulating factor, IL-6, IL-8, complement factor H, tumor necrosis factor-α, and α2 macroglobulin) in CSF are greatly reduced.
Table 1. Chinese version of the Montreal Cognitive Assessment (MoCA)
Table 2. International HIV Dementia Scale (IHDS)
Table 3. Abbreviations of CSF cytokines
Table 4. Analysis of cytokine profiles in CSF between the two groups of patients at WO and W24

Thirty-two cytokines were analyzed before and after lenalidomide treatment in the CI group and CNI group. In the CI group, there was a significant decline in cytokine levels after treatment (GRO, IL-10, G-CSF, IL-6, IL-8, P all <0.001; CFH, P=0.002; TNF-α, P=0.002; and α-2-M, P =0.003) (actual values in Table 4)
Table 5. Clinical features of the 7 patients

All seven patients were Chinese men, with an average age of 37.0 (interquartile range [IQR] 33.0-39.0) years. Of the seven patients, 85.7% had at least a junior high school education. Most of the patients (71.4%) were treated with an antiviral regimen containing integrase inhibitors. The average CD4 count was 151.5 (119.3-239.0) cells/uL. The median HIV RNA copy number was 0 (0-447.0) copies/mL. The median time for cognitive dysfunction onset after ART initiation was 309.0 (265.0-694.0) days. The median time between ART initiation and lenalidomide initiation was 440.5 (310.0-710.5) days. Headache (four patients) and alteration of consciousness (four patients) were the most common clinical symptoms (Table 1). All patients received standard anticryptococcal treatment as mentioned in the Methods, and no pathogens were detected in the CSF. All patients had been treated with corticosteroids, but their cognitive function was not significantly improved. They had not been treated with corticosteroids in the last 6 months.
Table 6. Cognitive analysis and CSF profile analysis between the two groups of patients

After lenalidomide treatment, there was a significant decline in the CSF WBC (P=0.032), CSF protein (P=0.034), CSF albumin (P=0.034), and CSF IgG levels (P=0.050) in the CI group. Spearman's correlation analysis revealed that the decrease in CSF albumin level (R2: −0.757, P=0.049) and increase in CSF glucose level (R2: 0.883, P=0.008) had a strong positive correlation with MoCA scores.

Table 7. Cognitive analysis and CSF profile analysis between the two group in WO and W24 (actual value)

The same as in Table 6, specific values are listed

Interpretation of results: The critical score is 26 points, and for those with education below primary school. 1 point is added to the original score.

TABLE 2
International HIV Dementia Scale.
International HIV Dementia Scale (IHDS)

Memory-Registration - Give four words to recall (dog, hat, bean, red) - 1 second to
say each. Then ask the patient all four words after you have said them. Repeat words
if the patient does not recall them all immediately. Tell the patient you will ask for
recall of the words again a bit later.
1. Motor Speed: Have the patient tap the first two fingers of the non-dominant hand
as widely and as quickly as possible.
4 = 15 in 5 seconds
3 = 11-14 in 5 seconds
2 = 7-10 in 5 seconds
1 = 3-6 in 5 seconds
0 = 0-2 in 5 seconds
2. Psychomotor Speed: Have the patient perform the following movements with the
non-dominant hand as quickly as possible: 1) Clench hand in fist on flat surface. 2)
Put hand flat on surface with palm down. 3) Put hand perpendicular to flat surface on
the side of the 5thdigit. Demonstrate and have patient perform twice for practice.
4 = 4 sequences in 10 seconds
3 = 3 sequences in 10 seconds
2 = 2 sequences in 10 seconds
1 = 1 sequence in 10 seconds
0 = unable to perform
3. Memory-Recall: Ask the patient to recall the four words. For words not recalled,
prompt with a semantic clue as follows: animal (dog); piece of clothing (hat);
vegetable (bean); color (red).
Give 1 point for each word spontaneously recalled.
Give 0.5 points for each correct answer after prompting
Maximum - 4 points.
Total International HIV Dementia Scale Score: This is the sum of the scores on
items 1-3. The maximum possible score is 12 points. A patient with a score of ≤ 10
should be evaluated further for possible dementia.

TABLE 3
Abbreviations of CSF cytokine

| | |
|---|---|
| α2M | alpha 2-macroglobulin |
| Apo. AI | apolipoprotein AI |
| Apo. E | apolipoprotein E |
| Aβ42 | amyloid beta 1-42 |
| CFH | complement Factor H |
| FGF-2 | fibroblast growth factor -2 |
| G-CSF | granulocyte colony-stimulating factor |
| GM-CSF | granulocyte-macrophage colony-stimulating factor |
| GRO | growth-related oncogene |
| IFN-γ | interferon-γ |
| IL-1α | interleukin-1α |
| IL-1β | interleukin-1β |
| IL-10 | interleukin-10 |
| IL-12p40 | interleukin-12p40 |
| IL-12p70 | interleukin-12p70 |
| IP-10 | interferon gamma induced protein 10 |
| MCP-1 | monocyte chemotactic protein 1 |
| MIP-1α | macrophage inflammatory protein-1α |
| MIP-1ß | macrophage inflammatory protein-1β |
| PDGF-AA | platelet-derived growth factor-AA |
| pTau | phosphorylated tau |
| sCD40L | soluble CD40 ligand |
| tTau | total tau |
| TNF-α | tumor necrosis factor-α |

TABLE 4
CSF cytokine profile analysis between the two group inW 0 and W 24 (actual value)

| | Cognitive improvement group | | | | | | | |
|---|---|---|---|---|---|---|---|---|
| | P1-W 0 | P1-W 24 | P2-W 0 | P2-W 24 | P3-W 0 | P3-W 24 | P4-W 0 | P4-W 24 |
| α-2-M | 58956 | 10777 | 98548 | 4765 | 62536 | 3814 | 9349 | 2942 |
| apoA-I | 7139 | 6850 | 14351 | 683.32 | 12453 | 668.25 | 3025 | 788.79 |
| Apo. E | 4805 | 5683 | 12881 | 1764 | 7098 | 2487 | 2155 | 1873 |
| Complement C3 | 15743 | 10475 | 36994 | 4010 | 24662 | 818.22 | 1365 | 1909 |
| CFH | 17591 | 4617 | 25658 | 2381 | 21323 | 2275 | 5247 | 1603 |
| FGF-2 | 16.08 | 16.08 | 27.97 | 16.08 | 27.97 | 16.08 | 33.5 | 16.08 |
| G-CSF | 2514 | 22.02 | 519.74 | 3.57 | 552.28 | 3.57 | 117.52 | 5.99 |
| GM-CSF | 2.1 | 0.98 | 1.78 | 1.02 | 1.11 | 1.94 | 10.19 | 2.81 |
| IFN-γ | 1.31 | 1.31 | 1.85 | 0.7 | 1.26 | 0.66 | 21.1 | 1.6 |
| GRO | 1878 | 111.65 | 1372 | 63.2 | 313.51 | 32 | 2385 | 149.74 |
| IL-10 | 25.46 | 4.21 | 22.79 | 4.14 | 16.38 | 2.45 | 36.76 | 4.07 |
| IL-12p40 | 3.62 | 1.78 | 0.8 | 0.38 | 0.52 | 0.92 | 41.6 | 10.92 |
| IL-12p70 | 2.27 | 2.46 | 1.25 | 0.67 | 2.09 | 2.09 | 2.79 | 1.34 |
| PDGF-AA | 2.78 | 5.66 | 2.78 | 6.92 | 2.78 | 2.78 | 7.73 | 2.78 |
| IL-13 | 1.36 | 1.19 | 1.05 | 0.8 | 0.74 | 0.71 | 1.33 | 1.09 |
| sCD40L | 6.98 | 2.49 | 3.04 | 2.49 | 2.49 | 4.91 | 9.19 | 2.49 |
| IL-17α | 57.05 | 0.62 | 0.87 | 0.62 | 4.12 | 0.62 | 9.9 | 0.62 |
| IL-1α | 1.86 | 1.91 | 1.77 | 1.71 | 1.48 | 1.65 | 1.25 | 1.37 |
| IL-1β | 2.15 | 1.15 | 2.01 | 1.09 | 1.15 | 0.88 | 3.19 | 1.11 |
| IL-2 | 1.12 | 0.72 | 0.9 | 0.69 | 0.77 | 0.64 | 1.29 | 0.81 |
| IL-3 | 0.29 | 0.41 | 0.3 | 0.44 | 0.24 | 0.43 | 0.55 | 0.34 |
| IL-4 | 1.11 | 1.49 | 12.24 | 2.22 | 0.28 | 0.82 | 11.56 | 0.82 |
| IL-6 | 7715 | 12.8 | 3387 | 63.58 | 2791 | 2.85 | 164.34 | 2.85 |
| IL-8 | 788.78 | 175.42 | 904.74 | 164.92 | 332.94 | 64.25 | 1514 | 118.93 |
| IP-10 | 9890 | 12150 | 10595 | 4404 | 9030 | 3063 | 14536 | 12231 |
| MCP-1 | 1838 | 1624 | 2475 | 2268 | 3313 | 987.31 | 1408 | 923.8 |
| MIP-1α | 9.3 | 4.61 | 52.56 | 18.47 | 5.15 | 3.38 | 3.38 | 3.38 |
| MIP-1β | 21.91 | 23.79 | 41.68 | 31.7 | 33.2 | 9.58 | 13.45 | 5.86 |
| TNF-α | 22.25 | 7.46 | 22.25 | 6.82 | 12.37 | 2.46 | 66.53 | 8.22 |
| αβ42 | 1111 | 2364 | 885.51 | 708.02 | 1449 | 1573 | 409.02 | 693.92 |
| tTau | 628.5 | 1594 | 419.25 | 431.47 | 640.93 | 752.55 | 199.06 | 234.58 |
| pTau 181 | 19.03 | 61.43 | 39.49 | 38.37 | 33.1 | 42.96 | 15.41 | 19.12 |

| | Cognitive improvement group | Cognitive non-improvement group | | | | |
|---|---|---|---|---|---|---|
| | P5-W 0 | P5-W 24 | P6-W 0 | P6-W 24 | P7-W 0 | P7-W 24 |
| α-2-M | 2136 | 7109 | 9510 | 2778 | 1226 | 4331 |
| apoA-I | 901.43 | 2047.9 | 37504 | 833.9 | 1251 | 1214 |
| Apo. E | 972.01 | 5462 | 6016 | 2829 | 606.14 | 2633 |
| Complement C3 | 2882 | 4249 | 11684 | 2914 | 1973 | 1335 |
| CFH | 807.24 | 3699 | 2831 | 1253 | 679.74 | 2446 |
| FGF-2 | 13.73 | — | 38.82 | 13.73 | 13.73 | 48.91 |
| G-CSF | 3.9 | — | 15.61 | 2.84 | 3.9 | 2.84 |
| GM-CSF | 0.81 | 2.58 | 0.85 | 1.29 | 1.11 | 1.29 |
| IFN-γ | 0.7 | 1.67 | 0.66 | 1.01 | 0.82 | 0.66 |
| GRO | 7.86 | 117.5 | 105.16 | 44.05 | 18.59 | 73.45 |
| IL-10 | 2.45 | — | 2.69 | 2.45 | 2.45 | 3.7 |
| IL-12p40 | 0.8 | 1.65 | 1.21 | 1.13 | 0.7 | 0.52 |
| IL-12p70 | 0.98 | 2.69 | 0.98 | 1.25 | 1.25 | 1.44 |
| PDGF-AA | 2.78 | 6.89 | 10.73 | 2.9 | 2.78 | 3.99 |
| IL-13 | 0.99 | 1.91 | 0.99 | 0.99 | 1.29 | 0.92 |
| sCD40L | 2.49 | — | 10.11 | 2.49 | 2.49 | 2.49 |
| IL-17α | 1.48 | — | 0.64 | 1.13 | 1.82 | 0.62 |
| IL-1α | 1.25 | 3.37 | 1.77 | 1.95 | 1.73 | 1.42 |
| IL-1β | 1.13 | 2.16 | 1.03 | 1.03 | 0.84 | 1.13 |
| IL-2 | 0.77 | 1.44 | 0.72 | 0.7 | 0.72 | 0.74 |
| IL-3 | 0.5 | 0.82 | 0.49 | 0.52 | 0.54 | 0.3 |
| IL-4 | 0.59 | 2.71 | 3.29 | 2.22 | 0.41 | 0.49 |
| IL-6 | 2.85 | — | 10.97 | 2.85 | 2.85 | 2.85 |
| IL-8 | 21.29 | 234.05 | 97.56 | 62.23 | 93.68 | 171.82 |
| IP-10 | 331.24 | 13221 | 7336 | 3398 | 3997 | 9823 |
| MCP-1 | 1027 | 3992.12 | 1913 | 981.12 | 1165 | 3011 |
| MIP-1α | 3.38 | — | 10.37 | 3.38 | 3.44 | 5 |
| MIP-1β | 7.64 | 31.83 | 24.72 | 13.92 | 4.18 | 17.91 |
| TNF-α | 1.26 | 8.22 | 5.76 | 3.01 | 3.65 | 5.21 |
| αβ42 | 397.62 | 2453 | 2132 | 1168 | 1099 | 1285 |
| tTau | 89.99 | 1309.82 | 585.02 | 681.32 | 216.26 | 628.5 |
| pTau 181 | 20.75 | 85.14 | 64.31 | 48.8 | 27.67 | 36.34 |

α-2-macroglobulin (α-2-M),
apolipoprotein A-I (apoA-I),
Complement Factor H (CFH),
fibroblast growth factor 2 (FGF-2),
granulocyte-colony stimulating factor (G-CSF),
granulocyte-macrophage colony-stimulating factor (GM-CSF),
interferon-γ (IFN-γ),
growth-regulated oncogene (GRO),
soluble CD40 ligand (sCD40L),
platelet-derived growth factor-AA (PDGF-AA),
interleukin (IL)-12p40,
monocyte chemoattractant protein-1 (MCP-1),
macrophage inflammatory protein-1α (MIP-1α),
tumor necrosis factor-α (TNF-α),
amyloid-β (1-42) (αβ42),
total Tau (tTau),
and phosphorylated tau 181 (pTau181)

TABLE 5
Clinical features of the 7 patients

| Characteristics | Baseline value* | |
|---|---|---|
| Age (year ) | 37.0 | (33.0-39.0) |

Sex No (%)

| | | |
|---|---|---|
| Male | 7.0 | (100) |
| Female | 0 | (0) |

Education No (%)

| | | |
|---|---|---|
| Primary | 1.0 | (14.3) |
| Middle school | 5.0 | (71.4) |
| High school | 1.0 | (14.3) |
| Graduate and above | 0 | (0) |
| BMI (kg/(m)\hat{ }2) | 20.8 | (19.8-27.6) |
| Time from anti-CM to ART initiation, days | 36.5 | (25.0-40.0) |
| Time from anti-CM to LEN, days | 477.0 | (338.8-746.8) |
| Time from ART initiation to LEN, days | 440.5 | (310.0-710.5) |
| Time from ART initiation to cognitive | 309.0 | (265.0-694.0) |
| decline, days | | |

ART regimen No (%)

| | | |
|---|---|---|
| 2NRTI + 1NNRTI | 1 | (14.3) |
| 2NRTI + 1PI | 1 | (14.3) |
| 2NRTI + 1 INSTI | 5 | (71.4) |

CD4 cell count (Screen) No (%)

| | | |
|---|---|---|
| <200 cells/ul | 6 | (85.7) |
| 200-500 cells/ul | 1 | (14.3) |
| >500 cells/ul | 0 | (0) |
| CD4 cell count (Screen) | 151.5 | (119.3-239.0) |
| HIV RNA | 0 | (0-447.0) |

Clinical presentation No (%)

| | | |
|---|---|---|
| Fever | 2 | (28.6) |
| Headache | 4 | (57.1) |
| Alteration of consciousness | 4 | (57.1) |
| Seizures | 1 | (14.3) |

*The values are either medium (range) or # (%)
ART: antiretroviral therapy;
CM: cryptococcal meningitis;
LEN: Lenalidomide;
NRTI: nucleoside reverse transcriptase inhibitor;
NNRTI: non-nucleoside reverse transcriptase inhibitor;
INSTI: Integrase strand transfer inhibitor;
PI: protease inhibitor

TABLE 6
Cognitive Analysis and CSF Profile Analysis Between the Two Groups

| | | | | | | ^{Δ}MoCA | | ^{Δ}IHDS | |
|---|---|---|---|---|---|---|---|---|---|
| | | | Cognitive CNI | | | Spearman's | | Spearman's | |
| | Cognitive CI Group | | Group | | P-value | Correlation^{#a} | P-value | Correlation^{#b} | P-value |
| ^{Δ}MoCA (scores) | 8.5 | (3.3-24.3) | 1.0 | (1.0-3.0) | 0.154 | | | 0.505 | 0.248 |
| ^{Δ}IHDS (scores) | 5.25 | (1.0-9.3) | 1.5 | (1.3-2.0) | 0.480 | 0.505 | 0.248 | | |
| ^{Δ}ICP (mmH₂O) | 10.0 | (−57.5-41.0) | −20.0 | (−57.5-5.0) | 0.858 | 0.282 | 0.540 | 0.793 | 0.033 |
| ^{Δ}WBC (×10⁶/L) | −118.0 | (−179.0-64.0) | 7.0 | (4.0-7.0) | 0.032 | −0.264 | 0.568 | −0.054 | 0.908 |
| ^{Δ}ALB (g/L) | −299.2 | (−302.5-166.1) | 0.4 | (−1.9-5.2) | 0.034 | −0.757 | 0.049 | −0.679 | 0.094 |
| ^{Δ}IgG (g/L) | −186.8 | (−227.9-100.5) | 24.3 | (11.9-28.6) | 0.050 | −0.580 | 0.228 | −0.600 | 0.208 |
| ^{Δ} Protein (g/L) | −4.4 | (−5.6-2.5) | 0.3 | (0.1-0.4) | 0.034 | −0.595 | 0.159 | −0.679 | 0.094 |
| ^{Δ}Chlorine (mmol/L) | 2.0 | (1.5-7.5) | −4.0 | (−4.0-2.5) | 0.208 | 0.110 | 0.814 | 0.145 | 0.756 |
| ^{Δ}Glucose (mmol/L) | 0.4 | (0.2-0.7) | −0.3 | (−0.4-0.2) | 0.077 | 0.883 | 0.008 | 0.536 | 0.215 |

^{#a}This column showed Spearman's correlation between ^{Δ}MoCA and each of the other Δ value
^{#b}This column showed Spearman's correlation between ^{Δ}IHDS and each of the other Δ value
^{A}= week 24-week 0;
CI group: the cognitive improvement group;
CNI group: the cognitive non-improvement group;
MoCA: Montreal Cognitive Assessment scores;
IHDS: International HIV Dementia Scale scores;
ICP: intracranial pressure; WBC: white blood cell;
ALB: Albumin

TABLE 7
Cognitive analysis and CSF profile analysis between the two group in W 0 and W 24 (actual value)

| | Cognitive improvement group | | | | Cognitive non-improvement group | | |
|---|---|---|---|---|---|---|---|
| | Patient 1 | Patient 2 | Patient 3 | Patient 4 | Patient 5 | Patient 6 | Patient 7 |
| MoCA-W 0 | 26 | 13 | 0 | 23 | 23 | 24 | 14 |
| MoCA-W 24 | 30 | 26 | 28 | 26 | 24 | 25 | 19 |
| IHDS-W 0 | 8 | 2.5 | 0 | 12 | 9.5 | 10.5 | 9 |
| IHDS-W 24 | 10 | 11 | 10 | 12 | 12 | 12 | 10 |
| ICP-W 0(mmH₂O) | 150 | 80 | 140 | 210 | 220 | 110 | 190 |
| ICP-W 24(mmH2_{O}) | 120 | 152 | 150 | 85 | 200 | 120 | 95 |
| WBC-W 0((×106/L) | 68 | 120 | 20 | 240 | 0 | 3 | 0 |
| WBC-W 24((×106/L) | 2 | 2 | 10 | 0 | 1 | 10 | 7 |
| ALB-W 0(g/L) | 296 | 352 | 341 | 85.6 | 27.3 | 33.5 | 58.8 |
| ALB-W 24(g/L) | 69.7 | 52.8 | 35.3 | 52.7 | 23.2 | 43.4 | 59.2 |
| IgG-W 0(g/L) | 123 | 314 | 198 | 24.5 | 3.86 | 4.67 | 10.1 |
| IgG-W 24(g/L) | — | 45.1 | 11.2 | 10.4 | 3.35 | 29 | 43 |
| Protein-W 0(g/L) | 4.67 | 7.98 | 5.19 | 1.317 | 0.43 | 0.56 | 0.76 |
| Protein-W 24(g/L) | 1.081 | 1.24 | 0.745 | 0.788 | 0.37 | 0.908 | 1.15 |
| Chlorine-W 0(mmol/L) | 119 | 118 | 127 | 119 | 127 | 118 | 127 |
| Chlorine-W 24(mmol/L) | 128 | 131 | 128 | 121 | 123 | 127 | 123 |
| Glucose-W 0 (mmol/L) | 1.8 | 2.1 | 2.6 | 2.7 | 5 | 2.8 | 2.8 |
| Glucose-W23 (mmol/L) | 2.4 | 2.5 | 3.6 | 2.6 | 4.7 | 2.4 | 2.8 |

MoCA: Montreal Cognitive Assessment scores;
IHDS: International HIV Dementia Scale scores;
ICP: intracranial pressure; WBC: white blood cell;
ALB: Albumin

What is claimed is:

1. A method for improving or treating cognitive dysfunction caused by human immunodeficiency virus (HIV)-associated cryptococcal meningitis (CM) immune reconstitution inflammatory syndrome (IRIS) in a patient with lenalidomide, comprising administering a therapeutically effective amount of lenalidomide to the patient.

2. The method according to claim 1, wherein lenalidomide is administered for 6 treatment cycles, with each cycle comprising 21 days of lenalidomide administration followed by 7 days of no lenalidomide.

3. The method according to claim 1, wherein the dosage of lenalidomide is 25 mg/day.

4. The method according to claim 1, wherein the treatment with lenalidomide is carried out on the basis of continuing antiretroviral therapy (ART) and anti-cryptococcal treatment.

5. The method according to claim 4, wherein the median time between ART initiation and lenalidomide initiation is 310.0-710.5 days.

6. The method according to claim 1, wherein the treatment is directed to patients who have cryptococcosis diagnosed before ART; have completed 2 weeks of induction and 8 weeks of consolidation treatment or are negative for two times of cerebrospinal fluid (CSF) cryptococcus culture; or have plasma HIV-1 RNA of less than 500 copies per milliliter; and patients with chronic inflammation of the central system.

7. The method according to claim 1, wherein the patients with chronic inflammation of the central system have one or more of: CSF protein of higher than 0.45 g/L; CSF nucleated cell count of greater than 8/uL; lesions in the head indicated by abnormal signals in radiological examination, such as inflammation and edema.

8. The method according to claim 1, wherein the treatment is not useful for patients treated with immunosuppressants or other immunomodulators or cytotoxic drugs within 6 months.

9. The method according to claim 1, wherein the treatment is not useful for patients with severe underlying diseases of the heart, brain, liver, and kidney.

10. The method according to claim 1, wherein the treatment is not useful for patients with an absolute neutrophil count of 1000 cells/uL or less and a platelet count of less than 75,000/uL.

11. The method according to claim 1, wherein before the treatment with lenalidomide, the patients receive antifungal treatments, complete the initial therapy of CM with amphotericin B (AmB) and 5-flucytosine (5-FC), and have negative CSF fungal cultures; and then patients are continuously treated with fluconazole (FLU), in which the induction treatment comprises AmB (0.7-1.0 mg/kg)+5-FC (100 mg/kg bid) for 2 weeks; the consolidation treatment comprises 800 mg FLU for 8 weeks, and the maintenance treatment comprises 200 mg FLU daily.

12. The method according to claim 1, wherein the treatment reduces leukocytes, proteins, albumin, IgG and inflammatory cytokines in CSF; and the inflammatory cytokines comprises growth-related oncogene, interleukin [IL]-10, granulocyte-colony stimulating factor, IL-6, IL-8, complement factor H, tumor necrosis factor-α, and α2 macroglobulin.
